(19) United States
(12) Patent Application Publication
(10) Pub. No.: US 2019/0035040 A1
(43) Pub. Date: Jan. 31, 2019

(54) Method, Apparatus and System for Dynamic Analysis and Recommendations of Options and Choices based on User Provided Inputs

(71) Applicant: Eldermatics Inc., San Jose, CA (US)

(72) Inventor: Kumar Brijmohan Goel, Kumar, CA (US)

(73) Assignee: Eldermatics Inc., San Jose, CA (US)

(21) Appl. No.: 16/048,271

(22) Filed: Jul. 28, 2018

Related U.S. Application Data
(60) Provisional application No. 62/539,489, filed on Jul. 31, 2017.

(51) Int. Cl. G06Q 50/22; G16H 10/60; G06Q 40/08; G06F 15/18

(52) U.S. Cl. CPC G06Q 50/22; G06F 15/18; G06Q 40/08; G16H 10/60

(57) ABSTRACT

An option and choice framework method, apparatus and system is presented. It is a decision-support tool to help a user decide which option and coverage choice is best suited for his or her needs, from a multitude of available options or choices. The analysis of each option is based on responses provided by the user to various questions. Suitability of an option is based on a weighted score of questions and responses, mapped to various options, as well as an analysis of the options suitability (pros and cons) based on responses. The tool is dynamic in nature, as more options as well as more user inputs can be added to the tool over time.

### BENEFIT OF EARLIER FILING DATE FOR PRIORITY

This non-provisional patent application claims benefit of priority date through specific reference to provisional patent application No. 62/539,489 dated Jul. 31, 2017 under 35 U.S.C. 119 (e)(1). See also 37 C.F.R. 1.78.

### FIELD OF THE INVENTION

The present invention relates generally to informed consumer choice and buying. More specifically, the present invention proposes a method, apparatus and system to provide a framework (framework system) to a user to input his or her preference and options on available health insurance for providing him or her the best set of options and plans. While in this embodiment, the framework is applied to health insurance choice, the framework can be extended to any such choice and option exercise for the benefit of the user.

### BACKGROUND OF THE INVENTION

This algorithmic framework is a decision-support tool to help a user select better choices based on his/her needs. In the modern world, choices are getting increasingly complicated and too often customers do not have the knowledge or expertise to make the selections best suited for their needs and goals. Good examples are insurance, mutual funds, housing, automobiles, consumer durables, medical equipment and healthcare facilities.

Current solutions which are available neither take a user's needs and preferences into account nor provide detailed analysis or recommendations. The user is left to either go through comments left by other users on social media and e-commerce sites or ask friends and family. Occasionally, a “yes or no” decision-tree is available which falls woefully short of understanding full set of user's needs. None of these options takes a user's needs fully into account or provides analysis and appropriate recommendations.

The problem of “decision-support for right-fit selection by end user” is encountered in many situations. While many solutions are feasible, the emphasis is on creating a solution which is simple, effective, user friendly and which would be applicable across many products and services. This invention presents a solution for these issues and has the sought benefits.

### BRIEF DESCRIPTION OF THE DRAWINGS

FIG. 1 is one embodiment of a first set of questions (interrogatories) asked from a user for personal details applicable to choosing health insurance coverage. Particularly, preferred plan, familiarity with plan, sex, and tobacco consumption, date of birth and use of any prescription drugs are sought.

FIG. 2 is one embodiment in an exemplary way of a second set of questions (interrogatories) from a user regarding his or her situation which may impact choice of health coverage. Specific information is sought from any healthcare benefit sources, drug coverage, applicability of special situations including recent loss of insurance coverage and the effective date of the said situation are sought.

FIG. 3 is one embodiment of a third set of questions (interrogatories) asked from a user regarding the health insurance coverage he needs or desires. State of health, special needs, preference of a network, additional coverage and travel profile is determined through questions.

FIG. 4 is one embodiment of a first set of response from the framework tool to questions asked in FIG. 1, FIG. 2 and FIG. 3. Inputs from the different sets of questions are used to present analysis of available options (types of health insurance plans) and recommend the best-suited ones for the user (one recommended and one suggested, as applicable). User can review analysis provided for each option, click on embedded links to learn more and also look at system recommendations. Number of plans available for each type within each option is also provided. In a nutshell, the system provides the user information, guidance and analysis to decide which option or plan type will be best suited for the user's situation, needs and preferences. Options that are disabled based on inputs are shown to be non-selectable (disabled as choice).

FIG. 5 is an exemplary embodiment of a selected option chosen from FIG. 4 using information and analysis presented by the system based on inputs from the user entered through queries discussed for FIG. 1, FIG. 2 and FIG. 3. Health insurance plans corresponding to the chosen option are displayed to the user by type of plan. User may filter, compare and look at details of various plans. The selected plan(s) are popped up to the top where the user can look at his or her plan selection and proceed.

FIG. 6 is one embodiment of the flowchart used in the framework system showing inputs from the user to consolidated analysis, ranking of suitability, choice of plans, mechanisms to filter, compare and select plans.

FIG. 7 is one embodiment of the database accompanying the framework system where the keys to access are options for health plan, questions and responses. Weightage, response score, pros and cons of response, based on keys, are used to analyze and recommend options on health care plans.

FIG. 8 is an exemplary embodiment of the calculus involved in selecting the best option, taking into account the responses to question, accessing the corresponding pros and cons for the option, providing a score to responses (response score), weightage to questions (interrogatories) and presenting this analysis to the user based on his responses.

### DETAIL DESCRIPTIONS OF THE INVENTION

All illustrations of the drawings are for the purpose of describing selected versions of the present invention and are not intended to limit the scope of the present invention.

In the following description specific details are set forth describing certain embodiments. It will be apparent, however, to one skilled in the art, that the disclosed embodiments may be practiced without some of these entire specific details. The specific embodiments presented are meant to be illustrative, but not limiting. One skilled in the art may realize other material that, although not specifically described herein, is within the scope and spirit of this disclosure. For purposes of this disclosure, option and choice framework system may include any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, or other purposes. For example, framework system may be a hardware device of size, shape, performance, functionality, and price. In another embodiment, it may comprise of software components capable of being loaded to run on a hardware device. The framework system may include random access memory (RAM), one or more processing resources such as a central processing unit (CPU) or hardware or software control logic, read only memory (ROM), and/or other types of nonvolatile memory. Additional components may include one or more disk drives, one or more network ports for communicating with external devices as well as various input and output (I/O) devices, such as a keyboard, a mouse, and a video display. The framework system may also include one or more buses operable to transmit communications between the various hardware components. The framework system may be dedicated system of hardware and software. In another embodiment, it may constitute transferable software code loadable and runnable on a general purpose computer system.

Software, in accordance with the present disclosure, such as program code and/or data, may be stored on one or more machine readable mediums, including non-transitory machine readable medium. It is also contemplated that software identified herein may be implemented using one or more general purpose or specific purpose computers and/or computer systems, networked and/or otherwise. Where applicable, the ordering of various steps described herein may be changed, combined into composite steps, and/or separated into sub-steps to provide features described herein.

This is a decision-support tool to help a user decide which option/choice is best suited for his/her purposes, from a multitude of such options or choices which may be available. The analysis of each option is based on responses provided by the user to various questions. Suitability of an option is based on a weighted score of questions and responses, mapped to various options for suitability and fit. The tool is dynamic in nature. More options as well as user-inputs can be added to the tool to make it more sophisticated over time. In this embodiment, the framework system is used for choosing the best options for health care insurance plans. In another embodiment, it may be used for choice of regular insurance, choice of mutual funds, housing, automobiles, consumer durables, medical equipment and healthcare facilities.

This framework system provides analysis of each available option as well as recommendations based on a weighted score computed for each option. The weighted score is based on user input about his or her situation, needs and preferences as well as pre-assigned weightages.

In one embodiment, the tool provides analysis of each available option as well as recommendations based on a weighted score computed for each option. The weighted score is based on user input about his/her situation, needs and preferences as well as assigned weightages. In one embodiment, the weightages for the responses and questions may be assigned based on web scraping. In another embodiment, the allocation of response score and question weightage may be through big data analysis. In another embodiment, this may be achieved by supervised or un-supervised machine learning through pattern matching, or through deep learning via multiple layers of data patterns and algorithms sometimes also referred to as artificial intelligence. These tools, methods and techniques may also be used to compute estimated costs for specific treatments or out-of-pocket costs for the user. By taking these inputs, the framework system is capable of deeper analysis based on experiences of users in same situation as the subject of the tool over several years. Since the analysis in this embodiment becomes more sophisticated, the choices so obtained are optimal and learned from a perspective of the subject user as well as several users similarly situated with subject user.

In one embodiment, by deploying the mechanisms of machine learning, big data analysis, deep learning and artificial intelligence, instead of limiting the choice of the most suitable health plans to subject user based on his or her zip code, the framework system may be used to choose optimal zip codes to live in for most suitable health care plans. Such an analysis may span interstate boundaries and may incorporate information and data on the plans for each state. Where the subject user is advancing in age or otherwise concerned about health care, the choice of optimal and affordable health care plan may be the most important factor to decide where to live.

In one embodiment, the framework system may be used to recommend suitable healthcare facilities (hospitals for example), healthcare providers (doctors for example) with certain expertise and/or experience, and also health insurance plans based on a user's health conditions, prescription drugs, treatment history, accessibility to care and/or cost.

In one embodiment, by deploying the mechanisms of machine learning, big data analysis, deep learning and artificial intelligence, instead of limiting the choice of the most suitable health plans to subject user based on his or her zip code, the framework system may be used to choose optimal zip codes to live in for most suitable health care plans. Such an analysis may span interstate boundaries and may incorporate information and data on the plans for each state. Where the subject user is 65 or above in age, the choice of optimal and affordable health care plan may be the most important factor to decide where to live.

In one embodiment, the tool can help a user select the right health insurance plan based on his/her needs. The health insurance plan selection involves a complex eco-system with various complexities. Not all components work with each other and not all of them are equally applicable or suitable, depending on a user's situation and preferences. Further, there are many plans available for each component. In one embodiment, such components may be hospital visits, emergency visits, primary care physician visits, specialist visits and hospital stays. In one embodiment, components may be prescription drugs or supplementary plans. In one embodiment, hospital insurance may be classified as Plan A, medical insurance may be classified as Plan B, with Plan D classified as prescription drug plan. In one embodiment, Plan C may be another type of plan allowing private insurance companies to provide facets of a health insurance plan.

The tool can be configured based on questions to user about a particular health insurance plan eligibility, coverage needs and preferences, and mapped to available part combinations. Using the tool, the user can then be presented with analysis of each option as well as suitable recommendations. Once a part combination is selected, the user can be displayed various plans available for each part, which the user can filter, compare and select. The calculus for this operation is based on query questions, weightages assigned to questions, scores assigned to responses, pre-determined pros and cons for each option and pros and cons assigned to each response of the subject user.

Relevant information and plan attributes are accessed from the database and displayed based on options selected by the subject user. Responses entered by the subject user to questions are used to consolidate the pros and cons for each option. This is in addition to the information, links and general pros and cons for that option. Response scores are multiplied by the weightage for each question to tabulate a total score for each option. This consolidated information is used to display analysis for the selected option to the user and also provide recommendations. The recommendations are based on the top scoring options based on this methodology.

The framework can be used for various products and services as well as implemented using different technologies. In one embodiment the framework system can be focused on Medicare or any other health care plan. In another embodiment the framework system can be used on life insurance. In yet another embodiment, it can be used for right and properly priced car selection and another for selecting the right hospital for a procedure.

The framework can also be implemented using various technologies and mechanisms. In one embodiment it can be implemented using a spreadsheet tool. In another embodiment, it can be implemented using databases in the cloud and relevant web services. In another embodiment the framework is made easier to integrate by making it available in XML. In an exemplary embodiment the framework is advanced to make it more sophisticated by including crowdfunded expertise on a plurality of subjects.

In another embodiment, the framework is advanced by using machine learning, deep learning and artificial intelligence concepts. In one embodiment the framework system is advanced by extracting intelligence from public data, shared social media data and internet.

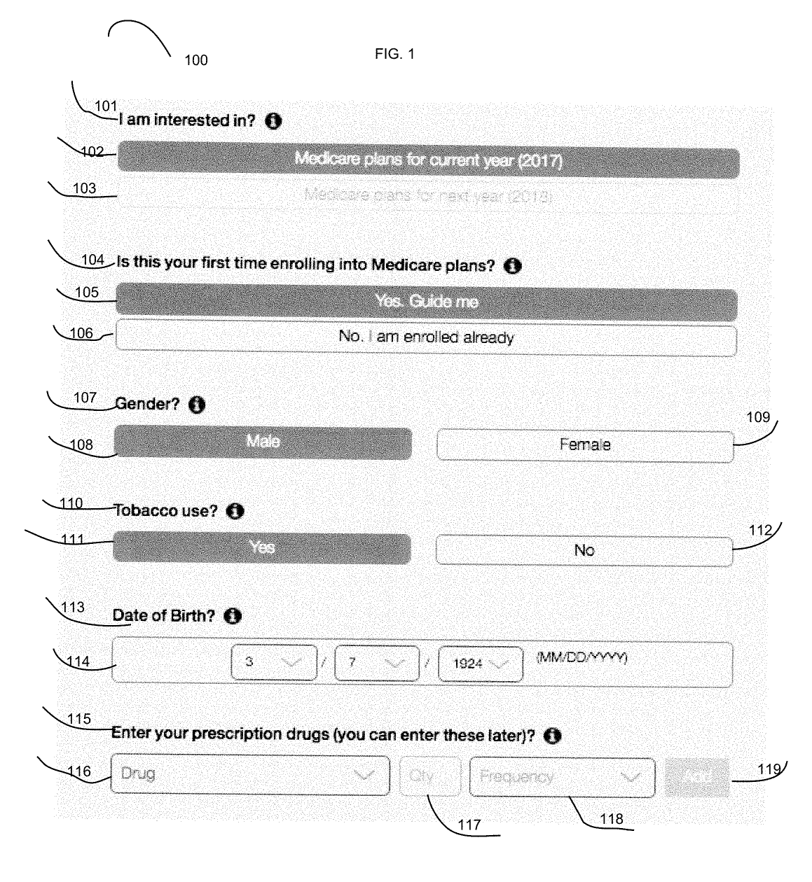

FIG. 1 100 illustrates the process with presentation of first set of questions 101 104 107 110 and 113. The first question enquires regarding year of interest, whether current or next year 102 103 with answer chosen by highlighting the proper rectangle 102. The familiarity with the plan is gauged through second question 104, with answer chosen through 105 and 106, if guidance in detail is needed 105 or the step can be skipped 106. In this case the user is later presented with an option to skip guidance and “show all” provision. The gender of the subject user is enquired and logged though 107 with answer logged as male 108 and female 109. The tobacco use is logged through question 110 with answers in 111 and 112. The date of birth question 113 is presented with answer logged as 114 in a standard month, day and year format. The focus here is with the personal information of the user.

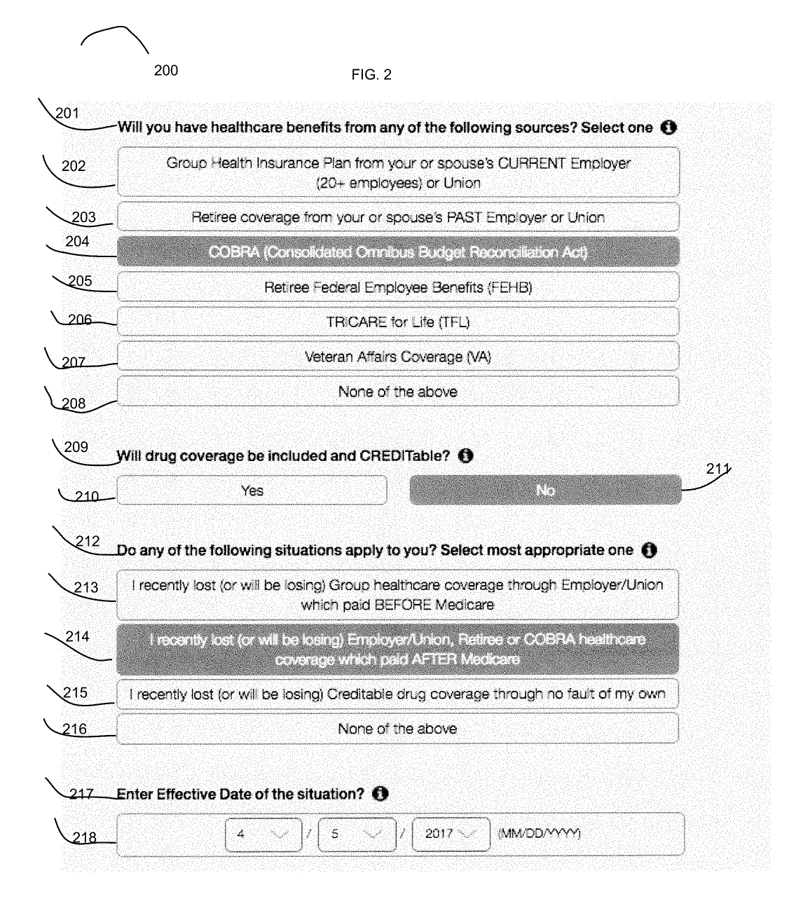

FIG. 2 200 is an exemplary illustration of a second set of questions presented to the subject user 201 209 212 217. Through first question in the third set 201, inquiry is made regarding any source of health insurance for the user, spouse's plan 202, retiree's coverage through union, employer or spouse 203, coverage under COBRA 204, federal retiree benefits 205, TRICARE 206, VA coverage 207 or none of these 208. Question regarding drug coverage 209 is presented with answers provided in 210 or 211. A third question 212 is presented regarding any special situations with answer recorded as lost coverage which was primary before Medicare 213, secondary after Medicare 214, loss of recent drug coverage 215 or none of these 216. Finally an effective date for chosen special situation is asked and logged as 218. The logged date is used to determine new plan eligibility. The inquiry focusses on the situation of the user with respect to insurance eligibilities.

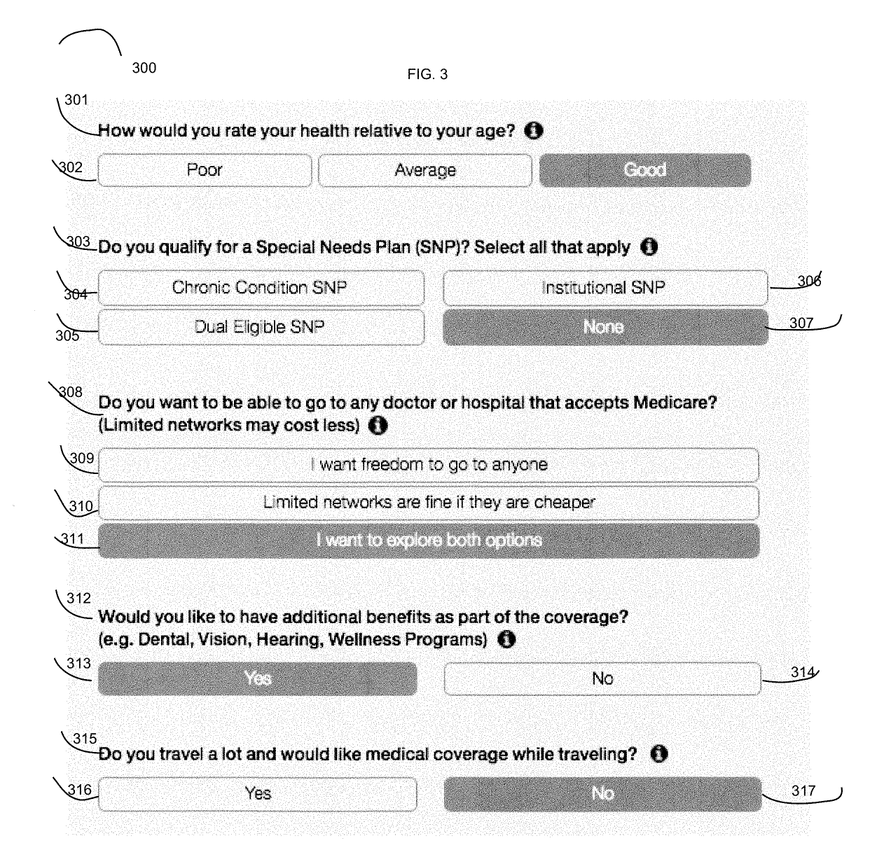

FIG. 3 300 is illustrative of presentation of a third set of questions to the subject user 301 303 308 312 315. The response to first question in this set are shown 302. Similarly the responses to question two 303 are shown 304 305 306 307. This question 303 deals with an inquiry if the patient has special needs. Question three 308 asks on whether broader network access is desired with possibility to pick broader, limited and both options 309 310 311. In one embodiment, the fourth question in the first set 312 enquires on added coverage for dental, vision, hearing or wellness programs with answers reflected as positive through 313 and negative through 314. Final question 315 enquires about travel profile of the subject user reflected as positive through 316 and negative through 317. The answers are recorded as part of the user access data, using the relational database access based on options and responses as above. The subject user is then presented with another set of questions or taken to the options page. The inquiry here attempts to determine user's preferences.

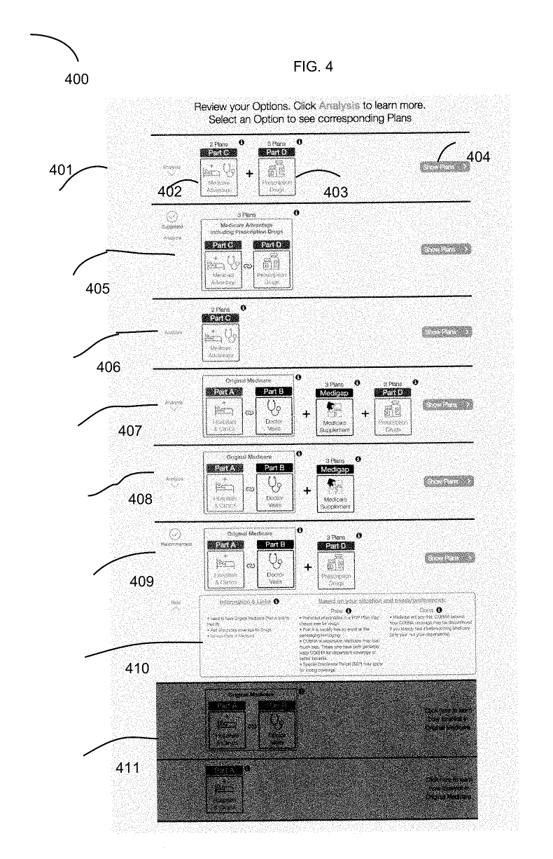

FIG. 4 400 is an exemplary illustration, in one embodiment, of the choice of insurance options offered by the framework system in considering responses to a plurality of sets of questions and performing internal calculus. The calculus involves access to database based on keys of questions, responses and option. Using scores on responses, weightage on questions and pros and cons analysis at option and each response level, a subset of plans are offered to the subject user 401 405 406 407 408 409. In one embodiment, the plan with highest calculus score is offered as recommended 409. A few others are offered including suggested 405. Detailed analysis options buttons 402 are made available to the user for each option to get a view of the calculus involved in the choice of the option offered. The plans are characterized as Part A, Part B, Part C 402, Part D 403 and Med. Supp. (Medicare Supplement) with nice graphics indicating hospital plans, medical plans, prescription drug plans and supplemental insurance. Plans that are not available to the user due to his options and answers to questions are disabled out 410 411. A select option button is provided for the user to see details of the plans corresponding to that option 404. This enables a review by the subject user and analysis on a per option basis. By providing this mechanism, the framework system hides the complexity and details from the user and ensures that the most suitable option and plan is not missed and inapplicable or sub-optimal plans are not chosen. The graphics friendly nature of this process makes it amenable to all subject users.

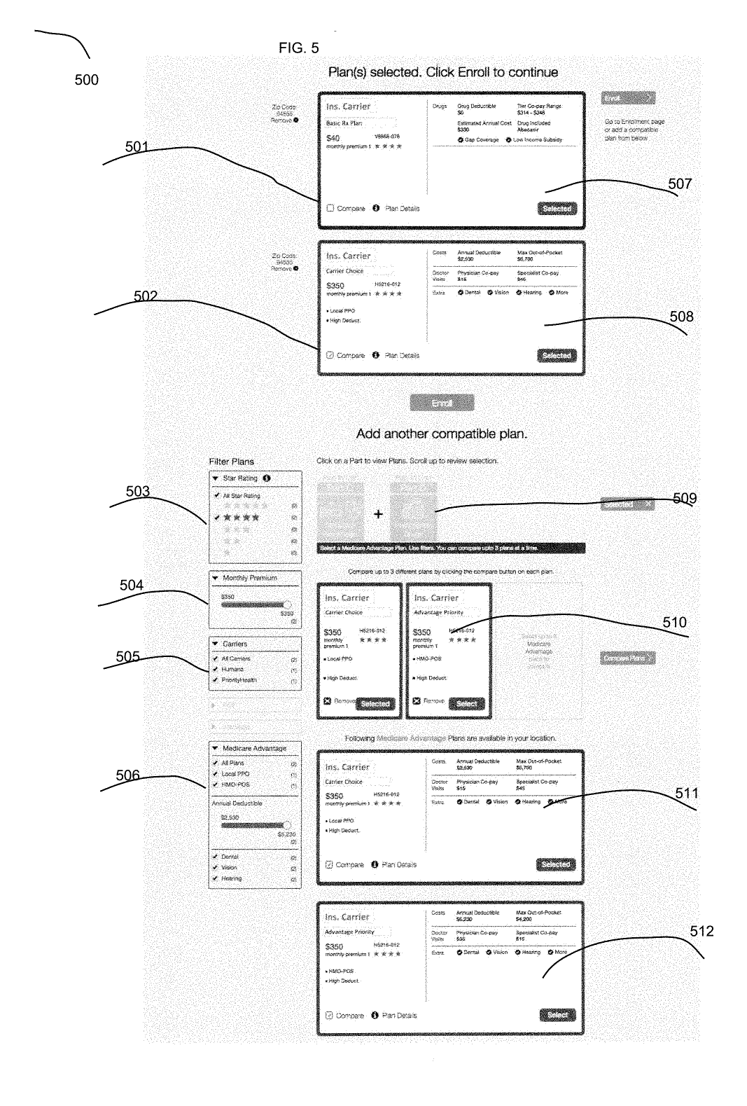

FIG. 5 500 is an exemplary illustration of the plans displayed to the subject user after the “show plans” icon is selected by the subject user from FIG. 4. The offered optimal plans are shown 511 512. These plans offer a select button. Once selected, the chosen plans bubble up 507 508. The insurance carrier is identified, along with cost and other important information along with symbols indicating Part C or D or Medicare Supplement. For multiple plan displays, several filters are provided to give option to subject user to choose and narrow down choices. Star Rating is shown 503. The monthly premium range is shown 504. The carriers are listed 505. For Plan C, as one embodiment, the Medicare plan C, both local PPO and HMOs plans are displayed and numbered by count 506. Range is provided for annual deduction. If chosen, the dental, vision and hearing plans are also shown 506. Once the subject user reviews the choices provided by the framework system calculus, option icons are provided to enroll in the selected plan or plans. The complexity of the user is severely reduced, as the subject user is involved solely in the presentation of his own facts and situation, the calculus takes care of identifying best choices as an expert tool, with easy user friendly interface.

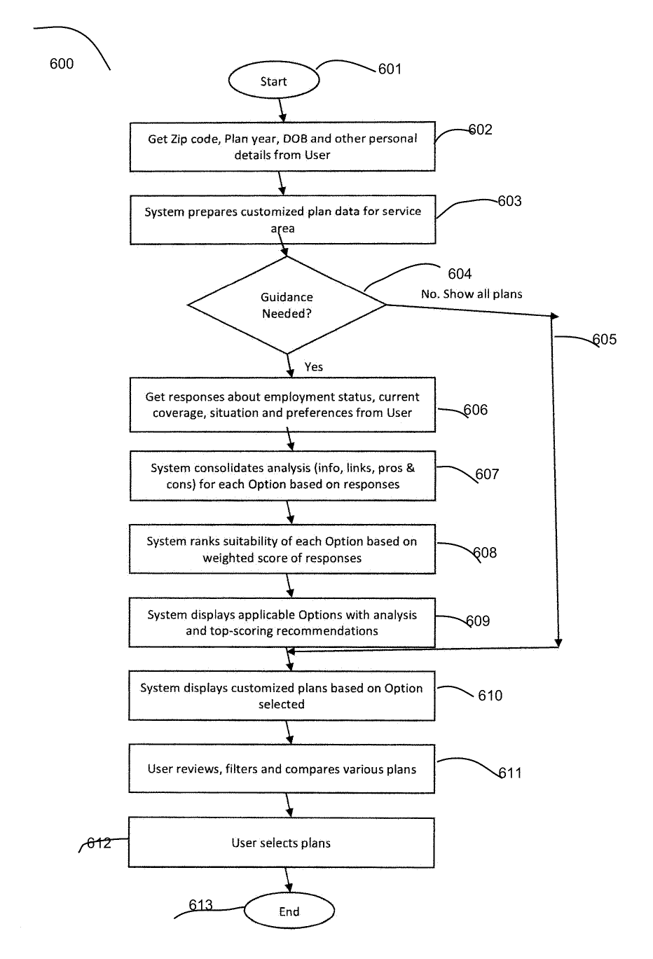

FIG. 6 600 is an exemplary illustration of the flowchart, indicating the steps involved in performing the framework calculus. The elliptical blocks 601 613 indicate start and stop of the algorithm. A rectangular box indicates actions or steps 602 603 606 607 608 609 610 611 612. A rhombus like quadrilateral indicates a choice of action based on an inquiry 604. As a first step, zip code, plan year, date of birth and other personal details are sought from the subject user by way of answers to simple questions 602. The framework system accesses plan data for the service area based on zip code 603. An inquiry is made if the user need guidance or it is veteran user 604. If the answer is in the affirmative, steps 606 607 608 and 609 are skipped 605. On the other hand if the answer is negative, the step 604 performs obtaining of responses about employment status, current coverage, situation and preferences 606. The system consolidates analysis by providing and considering information, links, pros and cons (things to consider) for each option based on responses 607. The framework system then ranks suitability of each option based on weighted scores of responses 608. The framework system then displays applicable options with analysis and top scoring recommendations 609. Irrespective of whether guidance is need or not, the framework system displays plans based on option selected on the type of coverage desired and other parameters 610. The framework system then offers opportunity to the subject user to review, filter and compare various plans 611. The process ends 613 with the subject user selecting plans 612 for application.

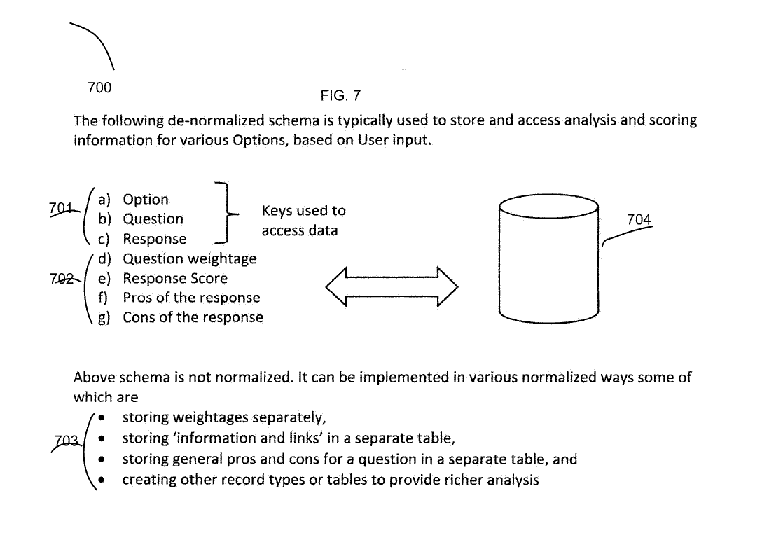

FIG. 7 700 is an exemplary depiction of the underlying mechanisms and databases involved in the framework system calculus. In one embodiment, a de-normalized schema is used to store and access analysis and scoring information for various options based on subject user input. In one embodiment, the keys used to access a relational database are coverage option, question and response 701. These keys are used to form a query into the relational database. The corresponding information stored in the relational database, in one embodiment are weightage for each question, score for each response, pros and cons of the coverage option and pro and cons of the response 702 (at least for most people). The relational database is stored in system memory 704. In one embodiment, the relational database 704 is capable of augmenting itself through inputs from independent processes involving artificial intelligence, machine learning, deep learning obtained through web scraping, social media sites and big data mining. In one embodiment, the database can be implemented as normalized through storing weightages separately. In another embodiment, the database 704 could be normalized by storing information and links in a separate table. In another embodiment, the pros and cons for a question could be stored in a separate table. In one embodiment, through use of artificial intelligence, machine learning, deep learning, big data analysis and other record types may be created to provide richer analysis. 703.

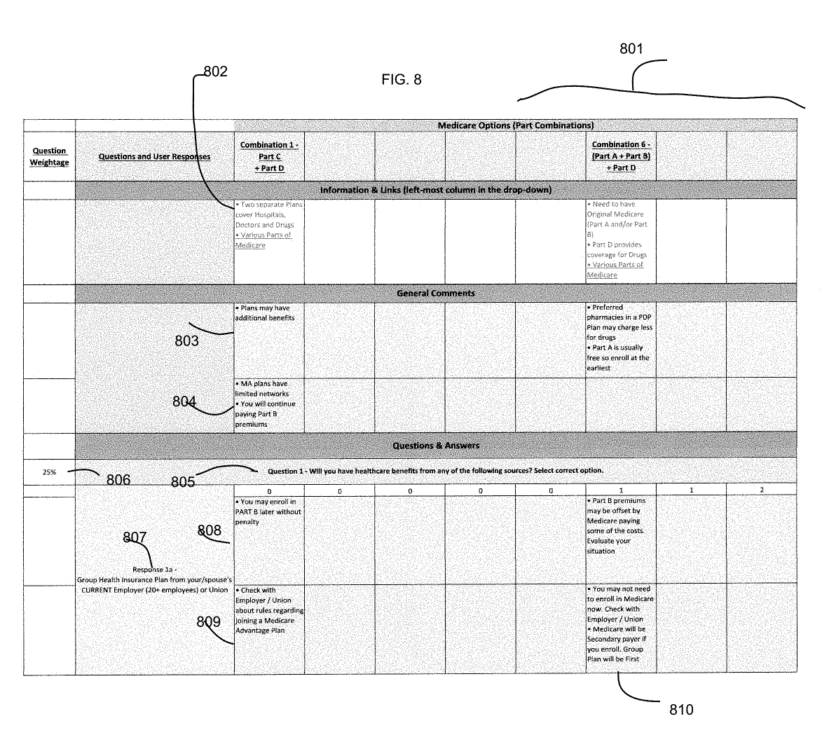

FIG. 8 800 illustrates the calculation method for the option favorability score on the basis of question weightage 806 and response score 805 along with considerations of pros 803 and cons 804 of the option itself and pros 808 and cons 809 per each response to each question. The various columns 801 in the figure represent various options and their structuring as one per column. Row 802 is utilized for providing information and clickable links to provide further details and information for each option. Row 811 provides general pros and cons for each option. Each option as well as question response can have several pros and cons. In an exemplary embodiment, 806 pertains to various questions which the user is asked about his needs and preferences. In the present example, the user is asked about health benefits from various sources, employer, spouse or union. Row box 807 represents the response of the subject user with the corresponding pros 808 and cons 809. The question weightage 806 is specified in first column. In one embodiment, the assigned weightage is 25%. In another embodiment, weightage can be fixed for each question or variable based on response. In FIG. 8, as one embodiment, it is fixed for each question. Column box 807 shows a response example. Each response to a question has its pros 808 and cons 809 for each option. The user is displayed both pros and cons based on the option selected 803 804 and responses provided to questions 808 809. In one embodiment, different words may be presented to the user, like “things to consider.” In one embodiment, a score assigned to every response 810, based on the option selected and its suitability for the user. Such a framework is used and made available for every option and for each relevant response to every question.

Embodiments as described herein as a framework system are exemplary. The examples provided above are illustrative only and are not intended to be limiting. For example, the framework system could be devised to handle auto, car and home insurance, mutual funds, housing, automobiles, consumer durables, medical equipment and healthcare facilities.

One skilled in the art may readily devise other systems consistent with the disclosed embodiments which are intended to be within the scope of this disclosure. Although the present invention has been explained in relation to its preferred embodiment of health care insurance (Medicare as an exemplary instance), it is to be understood that many other possible modifications and variations can be made without departing from the spirit and scope of the invention as herein described.

What is claimed is:

1. An option and choice framework system, comprising:
a plurality of computer systems with memory, peripherals, fixed and removable media, processor connected through buses or networks and;
a plurality of interrogatories to a user and;
a plurality of responses to the interrogatories and;
an allocation of a plurality of weightages to the interrogatories and;
an allocation of a plurality of scores to the responses and;
a use of the computer systems to execute a mathematical calculus to provide optimal choices and options to the user based on interrogatories, responses and their weightage and score.

2. The option and choice framework system of claim 1 where the choices and options are for a health insurance plan.

3. The option and choice framework system of claim 1 where interrogatories comprise the user's personal information, situational status on insurance eligibilities and preferences.

4. The option and choice framework system of claim 1 where response pros and cons are stored in a separate table.

5. The option and choice framework system of claim 1 where the mathematical calculus is implemented in a plurality of normalized ways.

6. The option and choice framework system of claim 1 where the mathematical calculus involves a use artificial intelligence, machine learning and big data analysis.

7. An option and choice framework apparatus, comprising:
a plurality of computer systems with memory, peripherals, fixed and removable media, processor connected through buses or networks and;
a plurality of interrogatories to a user and;
a plurality of responses to the interrogatories and;
an allocation of a plurality of weightages to the interrogatories and;
an allocation of a plurality of scores to the responses and;
a use of the computer systems to execute a mathematical calculus to provide optimal choices and options to the user based on interrogatories, responses and their weightage and score.

8. The option and choice framework apparatus of claim 7 where the choices and options are for a health insurance plan.

9. The option and choice framework apparatus of claim 7 where interrogatories comprise the user's personal information, situational status on insurance eligibilities and preferences.

10. The option and choice framework apparatus of claim 7 where response pros and cons are stored in a separate table.

11. The option and choice framework apparatus of claim 7 where the mathematical calculus is implemented in a plurality of normalized ways.

12. The option and choice framework apparatus of claim 7 where the mathematical calculus involves a use of any of artificial intelligence, machine learning and big data analysis.

13. An option and choice framework method, comprising:
a plurality of interrogatories to a user and;
a plurality of responses to the interrogatories and;
an allocation of a plurality of weightages to the interrogatories and;
an allocation of a plurality of scores to the responses and;
a use of the computer systems to execute a mathematical calculus to provide optimal choices and options to the user based on interrogatories, responses and their weightage and score.

14. The option and choice framework method of claim 13 where the choices and options are for a health insurance plan.

15. The option and choice framework method of claim 13 where interrogatories comprise the user's personal information, situational status on insurance eligibilities and preferences.

16. The option and choice framework method of claim 13 where response pros and cons are stored in a separate table.

17. The option and choice framework method of claim 13 where the mathematical calculus is implemented in a plurality of normalized ways.

18. The option and choice framework method of claim 13 where the mathematical calculus involves a use of any of artificial intelligence, machine learning and big data analysis.
